(19) United States
(12) Patent Application Publication
(10) Pub. No.: US 2021/0322000 A1
(43) Pub. Date: Oct. 21, 2021

(54) TISSUE RETRACTOR FOR RETRACTING INCISIONS

(71) Applicant: Cardiovascular Lab S.p.A.O Brevemente CV Lab S.p.A., Milano (IT)

(72) Inventors: Vincenzo GIAMBRUNO, Milano (IT); Giovanni ROLANDO, Milano (IT)

(21) Appl. No.: 17/260,680

(22) PCT Filed: Jul. 16, 2019
(86) PCT No.: PCT/IB2019/056056

§ 371 Date: Jan. 15, 2021

(30) Foreign Application Priority Data
Jul. 17, 2018 (IT) 102018000007267

(51) Int. Cl. A61B 17/02; A61B 17/34

(52) U.S. Cl. CPC A61B 17/0293; A61B 2017/3425; A61B 17/3423; A61B 17/0218

(57) ABSTRACT

A tissue retractor has a retractor body delimiting a central device opening and proximal and distal body ends. The proximal body end has a proximal flange having inner and outer proximal flange edges and a proximal flange body extending, with the retractor not delivered, away from the central device opening. The distal body end has a distal flange having inner and outer distal flange edges and a distal flange body extending, with the retractor not delivered, away from the central device opening. The retractor body has a retractor neck having a proximal end connected to the inner proximal flange edge and a distal end connected to the inner distal flange edge. The proximal flange has a proximal flange portion having concavity facing the distal flange, which has a distal concave flange portion having concavity, where the concavities are facing the same direction, and is dome segment-shaped or spherical crown-shaped.

### FIELD OF THE INVENTION

In its most general aspect, the present invention relates to a tissue retractor for retracting incisions, e.g. useful for accessing and creating a surgical area or site in an internal organ.

Furthermore, the present invention relates to a surgical access device comprising a tissue retractor and to an atrial retractor for cardiac surgery on a mitral or tricuspid valve comprising a surgical access device.

In particular, a field of application of the device is that of cardiac surgery, in particular MICS (Minimally Invasive Cardiac Surgery).

These devices are of particular interest for mitral or tricuspid valve surgery, e.g. for mitral valve repair or replacement, thus allowing the surgeon to access the heart through a short intercostal incision (minithoracotomy). However, the known solutions, once the heart is exposed, i.e. with bloodless heart and extracorporeal circulation, only allow the access to the interior of the left atrium through an incision which is mostly linear in the atrial ceiling, and the retraction of the edges of such an incision in the atrial ceiling.

The need is therefore strongly felt to support the inner tissues of the bloodless atrial ceiling which tend to close, in addition to retracting the edges of the incision of the atrial ceiling, in order to achieve visibility and ideal accessibility to the mitral valve throughout the duration of the operation.

It is the object of the present invention to obtain the retraction of the edges of the incision in the atrial ceiling in a simple and safe manner, and above all to obtain an optimal exposure of the mitral valve, thus minimizing the interferences with the operative phases of the surgery.

### BACKGROUND ART

Tubular devices of prevalent longitudinal extension are known from documents WO2016025715 to APPLIED MED RESOURCES, CN204306853U to SHENZHEN ANT HI TECH IND CO LTD, CN204072183U to ANHUI AOFU MEDICAL EQUIPMENT TECHNOLOGY CO LTD, EP2609880 to COVIDIEN LP, CN202751416U to HANGZHOU ZHIFANG MEDICAL TECHNOLOGY CO LTD, CN202637009 to TIAN KUIMING, CN102743199 to BEIJING HANGTIAN KADI TECHNOLOGY R & D INST, KR20120011240 to SR INNO MEDI CO LTD, EP1051111 to EDWARDS LIFESCIENCES CORP, GB1151993 to JOHNSON & JOHNSON, US2015209025 to ETHICON ENDO SURGERY INC, CN202263017 to HEIYING JIN, EP2457523 to TYCO HEALTHCARE, EP2229900 to TYCO HEALTHCARE, EP2238927 to ETHICON ENDO SURGERY INC, the main purpose of which tubular devices is to overcome a prevalent thickness of tissue and/or organs with respect to the desired lumen, thus resulting in long and extensible devices, but which do not care of protecting the incision edges or support them; on the contrary, they are mainly adapted to ensure a firm grip by facing their proximal and distal end edges towards the tissue in the form of clamps for gripping the tissue itself. On the contrary, the primary function of these devices is to be very adaptable to the walls of the incised tissue, with which they must work to maintain a fluid seal with the beating heart.

In particular, these known devices are unsuitable for protecting the edges of the tissue incision and are particularly unsuitable for vault-shaped tissues, such as those in the atrium of the human heart.

Retractor devices having a plurality of movable retractor blades are known from documents U.S. Pat. No. 7,981,031 to DEPUY SPINE, INC, US2011004067 to MARCHEK CONNIE, FRASIER WILLIAM, et al., EP2456373 to APPLIED MED RESOURCES. These known devices, although extremely flexible, comprise complex mechanisms which, especially if miniaturized, are very laborious to create, construct and use, making the surgery and surgeon's preparation complex.

Furthermore, these known devices are often traumatic for the margins of the tissue around the incision and not very suitable to contain the edges of the incision in a distributed manner and to support the tissue margins to ensure a free surgical site.

Finally, due to the large number of components which form them, they often make it difficult to fold and to deploy in situ.

In particular, the solution described in WO 2011/017440 A2 to the Mayo Foundation may only be used for a beating heart surgical application, i.e. for a heart full of blood and under pressure, and is designed specifically to be positioned in the apex of the ventricle (in WO 2011/017440 A2 the device is defined as “transapical port device”). This known device cannot be applied to atrial retraction in traditional or minimally invasive atrial surgery, with a still heart (i.e. empty or bloodless) and in extracorporeal circulation, because, for example, its design is aimed at being positioned on the ventricular ceiling, and is neither self-supporting nor adapted to keep the atrial tissue cut open.

This known solution, dedicated to beating heart surgery, has significant drawbacks when applied to an atrial bloodless heart operation. Of these, the following may be listed:

this known solution requires sealing systems which prevent losses of the under-pressure blood present in the ventricle, systems which would be traumatic for the tissue of the bloodless and therefore saggy atrium because the patient is in extracorporeal circulation and with heart not beating;

this known solution requires attachment/contrast systems to the heart muscle which avoid the expulsion of the device, which is in fact a valve holder, from its seat (the device of WO 2011/017440 A2 is sealed, like a cap, therefore exposed to the action of pressure from the interior of the ventricle to the exterior); WO 2011/017440 A2 in a described example describes the ability of the hooks to withstand pressures in the range of 300-400 mmHg;

in the figures of document WO 2011/017440 A2, the sealing and anti-ejection system function is entrusted to flanges made of conformable material; it is precisely because of such an ability of these flanges to conform, and thus adapt, to the internal walls of the ventricle (e.g. Cf. FIG. 16E of WO 2011/017440 A2) and to the external walls of the heart that such flange elements can perform the expected fluid sealing and anti-ejection function; this conformability of the flanges of this known solution does not lend itself at all to the walls of the atrium of a non-beating heart, which tend to sag and close on themselves, thus closing the surgical site, because they would simply adapt to such a tendency of the walls of the atrium making the flanges yield under the pressure of the saggy atrial tissues;

in the solution described in WO 2011/017440 A2 flanges are shown with silhouettes always in favor of conformability to the shapes of the heart muscle, in order to make them adhere thereto for sealing and anti-ejection purposes.

The surgical retractors of the prior art can all be referred to the approach of grasping one of the margins of the incision with a hook-like or blade-like instrument, and of pulling it laterally so as to open and widen the incision itself. These instruments are provided with a handle through which an operator performs the traction. The operator must remain passively by the patient's side until the operation is complete, sometimes hindering the surgical team. Adjustable supports have also been developed to hold the retractors in place avoiding the need for a dedicated operator, but these also create hindrance around the surgical site and are often difficult to adjust. These retractors offer a highly asymmetrical access to the surgical site; furthermore, they may also be traumatic for atrium tissues because of the extreme location of the tractive force application point.

Cylindrical or conical retractors have been recently suggested, such as the Superflex® from Fehling (stent type metal foil, rolled up to form an expandable cylinder) or the device described in document US2007/038032 A1 (expandable truncated cone obtained with a thin elastic foil). These devices, once inserted into the atrial incision and expanded, widen the margins of the incision itself and remain in place without recourse to an operator or a support. The surgical site thus obtained is regular in shape, but with a diameter not very different from that of the expanded incision, and once again all the tractive force is concentrated on the margins of the incision, with the respective risk of damage.

The need to overcome the limits described in the solutions of prior art remains strongly felt.

Furthermore, there is an increasing need to retract the margins of an atrial tissue incision, in particular of the vault-shaped myocardium in a bloodless heart, in a simple and above all safe manner by creating an opening or access to the internal parts of the organ to allow visibility and operation by the surgeon, without, however, excessively traumatizing the myocardium.

At the same time, the need is strongly felt for a surgical access device which has a limited number of components and which can be easily folded for insertion into the margins of the incision of the tissue while being capable of being easily extended in situ thus protecting the tissue itself.

### SOLUTION

It is an object of the present invention to provide a tissue retractor for retracting incisions and supporting the tissue edges having structural and functional characteristics such as to satisfy the aforementioned requirements and to overcome the drawbacks previously mentioned with reference to the discs of the prior art.

These and other objects are achieved by means of a tissue retractor according to claim 1, a surgical access device according to claim 59 and an atrial retractor according to claim 60.

Some advantageous embodiments are the subject of the dependent claims.

### DRAWINGS

Further features and advantages of the invention will be apparent from the description provided below of preferred embodiments thereof, given by way of non-limiting examples, with reference to the accompanying drawings, in which:

FIG. 1 shows a perspective view from the bottom of a tissue retractor according to the invention;

FIG. 2 illustrates a different side perspective view of the retractor in FIG. 1;

FIG. 3 shows a diagrammatic section of the left portion of a heart, in which the atrium with its atrial ceiling and the left ventricle are highlighted;

FIG. 4A shows a diagrammatic section view of the left heart portion in FIG. 3, in which an incision was made and the tissue retractor of the invention was delivered straddling the edge of the incision;

FIG. 4B shows the left heart portion in FIG. 4A with the tissue retractor viewed in axis with the longitudinal extension of the tissue retractor, thus highlighting the central retractor opening, or central device opening, and the wide access to the surgical site;

FIGS. 5 to 7 are axonometric views of three steps of the operations of folding the tissue retractor in a position with proximal flange raised and body in a contracted initial condition;

FIGS. 8, 9 and 10 also show axonometric views of three possible fully folded states or folded conditions of the tissue retractor with the folds in a “U”, “S” or “H” arrangement, e.g. which are useful for inserting it in the tissue incision;

FIGS. 11 and 12 show axonometric partial section views taken along a plane P passing through the longitudinal extension direction of the retractor and a radial direction orthogonal to this longitudinal direction of a tissue retractor according to a further embodiment;

figures from 13 to 18 show axonometric partial section views taken along said (longitudinal, radial) planes P of the retractor in FIGS. 11 and 12 in different steps of folding, inserted into an inserting and releasing device having a tubular body and a deposition plunger arranged straddling the tissue incision to be surrounded and retracted, when said tubular body of the inserting device is inserted into said tissue incision;

FIGS. 19 to 22 show axonometric views of a reinforcing element of the retractor drawn without the retractor body to highlight the details thereof and to show how the reinforcing element works when it is folded together with the body and inserted into the tissue incision;

FIGS. 23 to 25 show axonometric views of three different types of reinforcing petals of the reinforcing element adapted to support the retractor on the outer surface of the myocardium, or other tissue, or, with blades mainly extending in distal direction, for retracting the interior of the atrium or different chamber of the surgical site;

FIG. 26 shows an axonometric partial section view of a reinforcing petal of a reinforcing element which accommodates a reinforcing ring in a seat of the central portion thereof, thus leaving said reinforcing petal free to position itself in an appropriate angular position most adapted to reinforce the retractor body adapted to the specific anatomy of delivery;

FIG. 27 shows an axonometric view of a reinforcing ring which is adjustable in circumferential extension;

FIG. 28 shows in a perspective side view of a tissue retractor having its portions with mutually different strength and elasticity, in particular the flange proximal to the retractor neck and to the distal flange;

figures from 29 to 31 show views from the top or in axis with the longitudinal axis of extension of the retractor of a proximal flange and the delimited central device opening, in which the opening has a circular or ellipsoidal or oval section, thus allowing easier access and greater visibility to the specific pathology present in the surgical site;

FIGS. 32 to 33 show either views from the top or in axis with the longitudinal extension axis of the retractor of a proximal flange and the delimited central device opening, in which the flange also extends around the opening or in an asymmetrical manner;

FIGS. 34 to 36 show axonometric views of only the spherical-crown-shaped or bell-shaped distal flange and with an extension thereof either circumferentially uniform or non-uniform, e.g. to form a saddle or a “scalloped” shape, as well as the presence of inner discontinuities, e.g. such as windows adapted to allow access to a portion of the tissue wall to be treated while the portion of the surrounding tissue is retracted;

FIGS. 37 and 38 show a section on plane P of the retractor body illustrating two embodiments of the connecting portions of the proximal and distal flanges, thus allowing the creation, with the retractor neck, of a throat having a width, e.g. equal to the thickness of the atrial wall, or of the tissue to be retracted, or a “pocket” throat to contain a greater quantity of atrial tissue;

FIG. 39 shows a view of the retractor body in axis with the longitudinal axis of retractor body which shows longitudinal channels of the retractor body which locally reduce the thickness of the body and allow a predetermined folding order of the retractor body in a folded position;

FIG. 40 shows an axonometric view of a different embodiment of the retractor, in which the proximal flange has a thinner, therefore less rigid, sector e.g. placed in contiguity with the aortic valve, so as not to alter the coaptation thereof and to maintain the ability to administer a cardioplegia solution effectively without removing the retractor device;

FIG. 41 shows a perspective view of a tissue retractor, in which the presence of a reinforcing ring is shown in the portion thereof of retractor neck embedded within the retractor body is highlighted;

FIG. 42 shows a perspective view of a tissue retractor, in which the proximal flange has a number of openings, e.g. suited for suturing the flange to the tissue of the myocardium;

FIG. 43 shows a tissue retractor, in which the proximal flange has connecting or supporting or fixing ridges of surgical equipment or instruments, made as hooks or as connecting housings, e.g. snap-on;

FIG. 44 shows a section on a plane P of a tissue retractor, in which a lighting device is provided, e.g. a plurality of LEDs partially embedded in the retractor body and adapted to illuminate the interior of the retractor and therefore the surgical site;

FIGS. 45 and 46 show in a partially transparent axonometric section view taken along a plane P of a tissue retractor, in which the retractor neck portion has embedded therein a reinforcing ring which is adjustable in the circumferential extension thereof, together with an adjustment key which can be inserted into the retractor in a seat accessible in a direction parallel to the longitudinal direction of the retractor;

FIG. 47 shows a partial axonometric section view of a retractor, in which reinforcing elements are inserted in the form of longitudinally extending petals passing from the proximal flange to the retractor neck to the distal flange;

FIG. 48 shows a partial axonometric section view of a retractor, in which reinforcing elements are inserted in the form of longitudinally extending petals passing from the proximal flange to the retractor neck to the distal flange and in which said petals are connected to a reinforcing ring placed in the retractor neck;

FIG. 49 shows a tissue retractor in a longitudinal section (taken along axis X-X) according to a further embodiment, in which a portion of the proximal flange is shown enlarged and the surface of the proximal flange facing the tissue has a surface roughness given by flange surface reliefs.

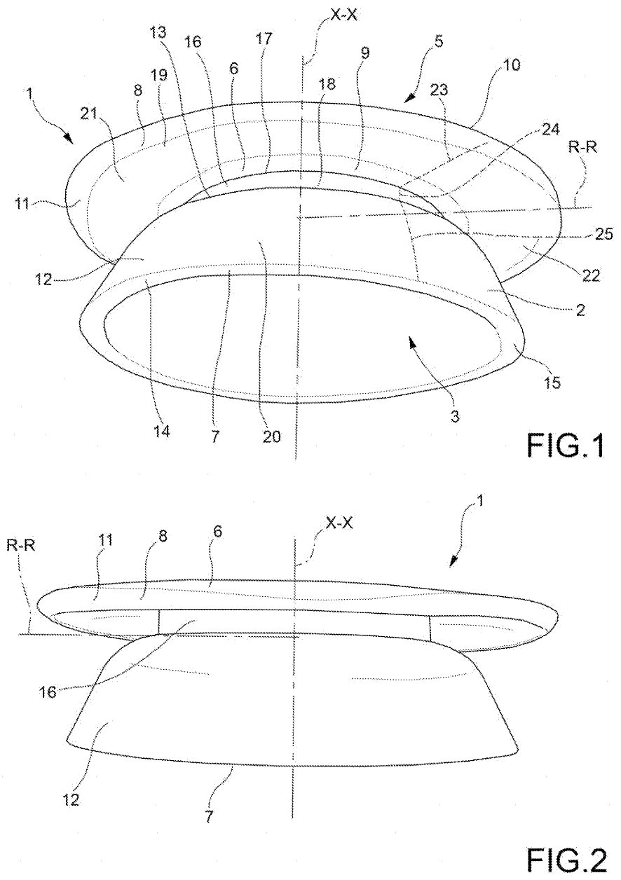

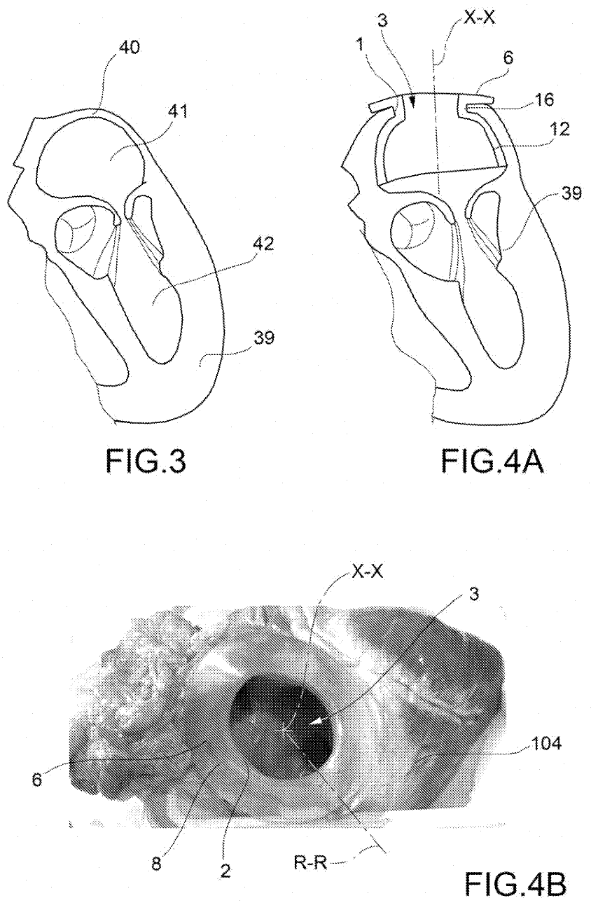

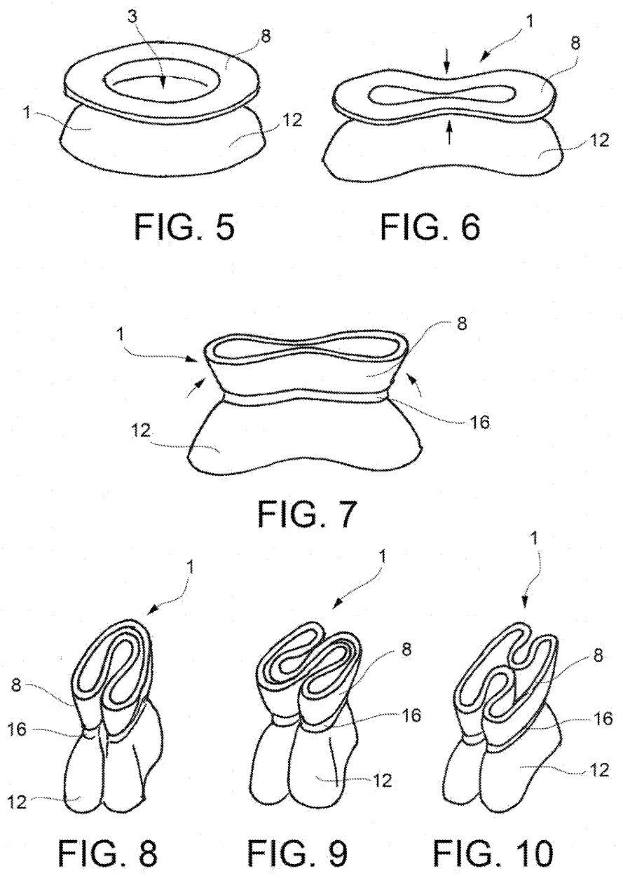

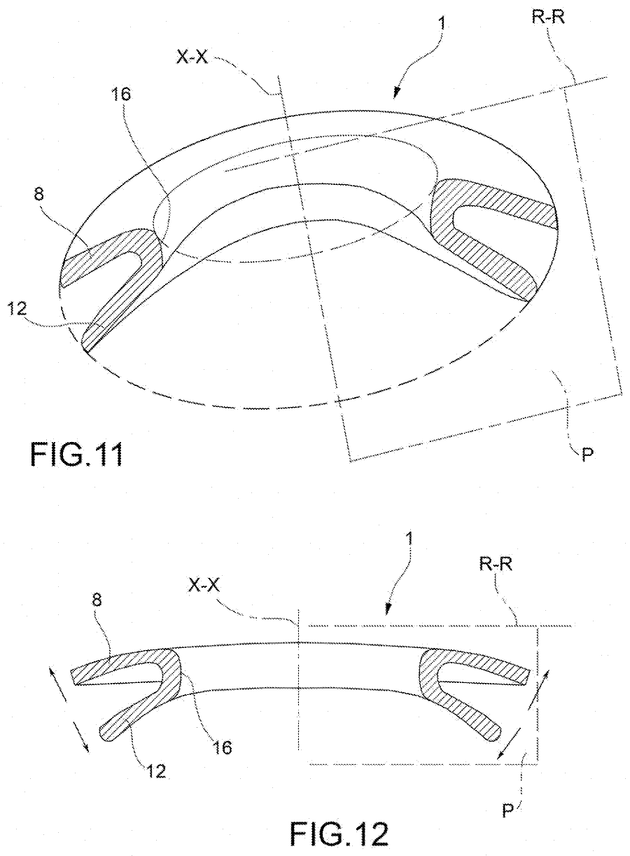

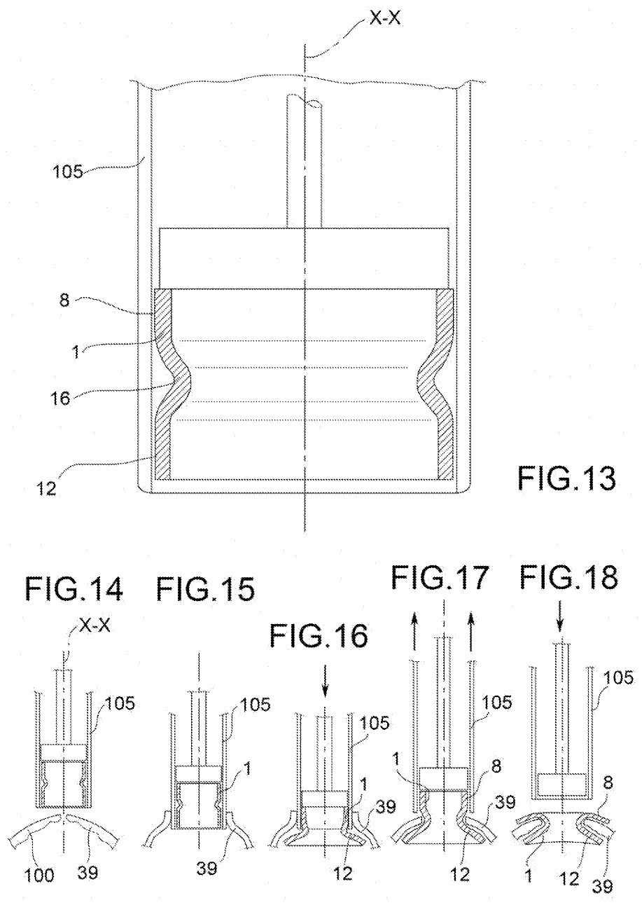

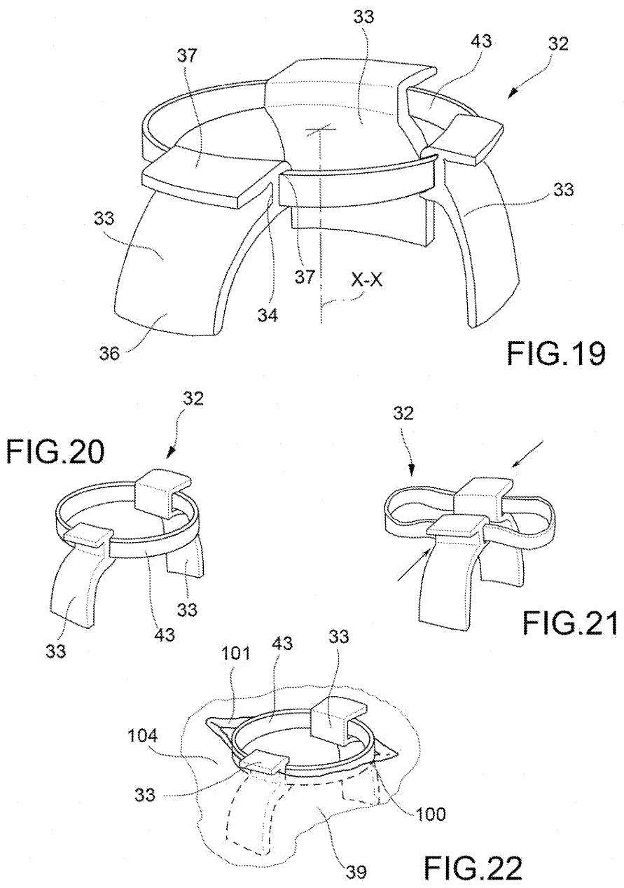

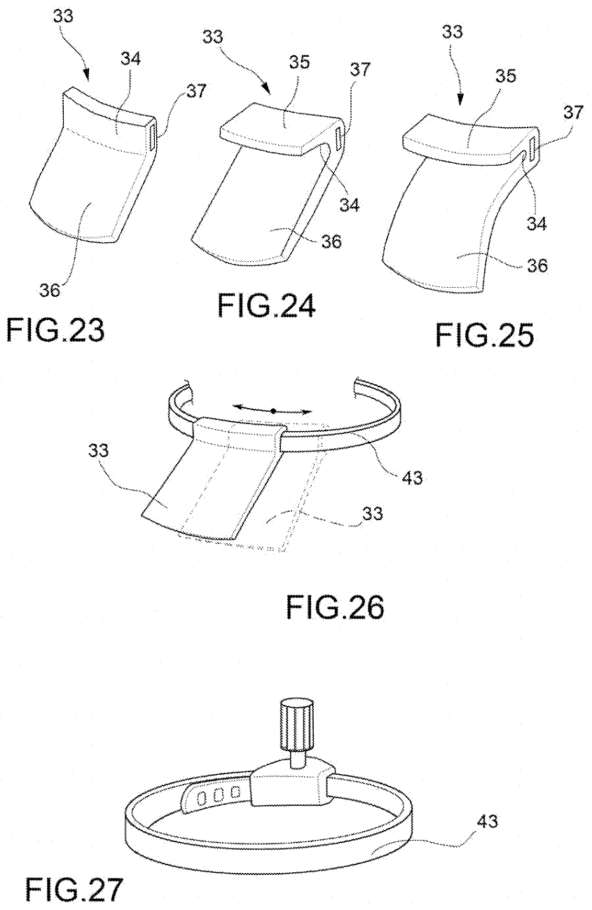

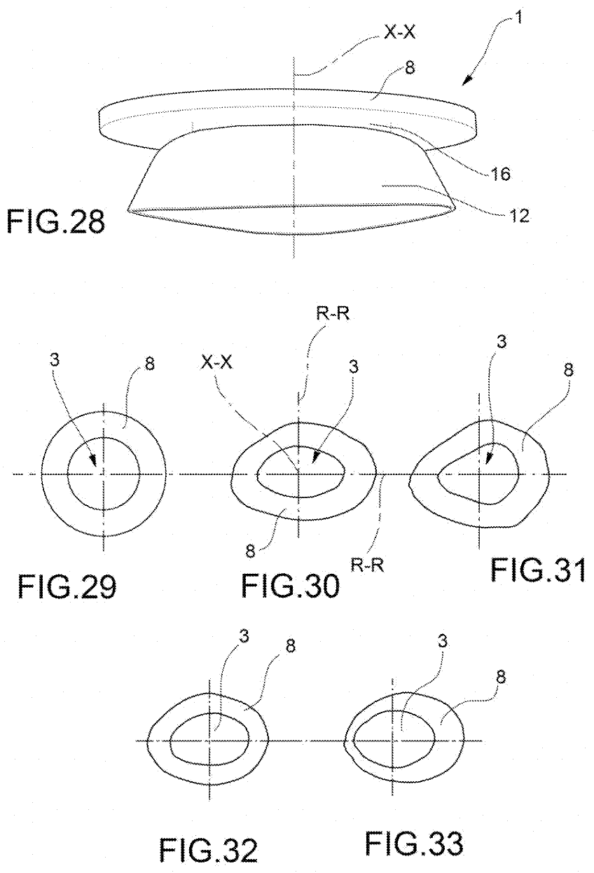

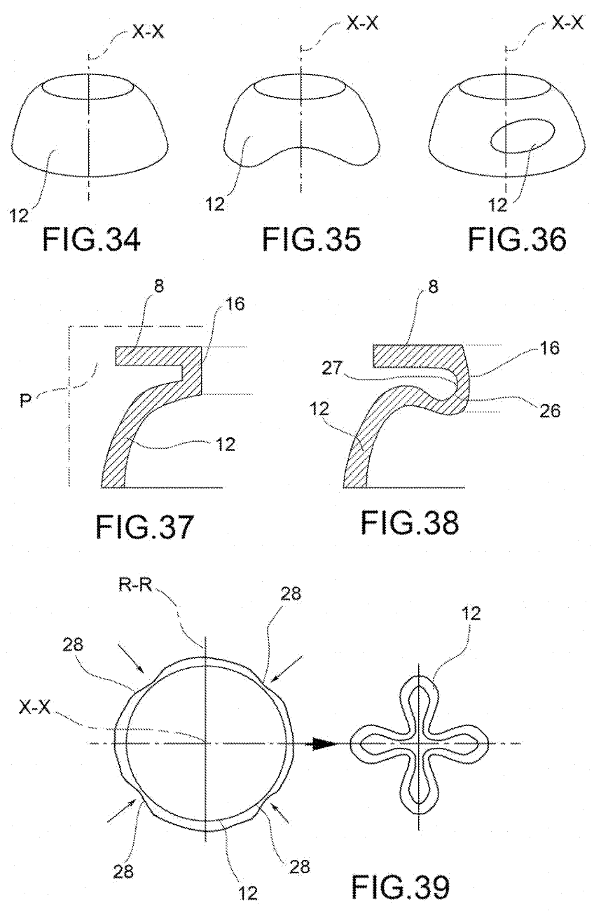

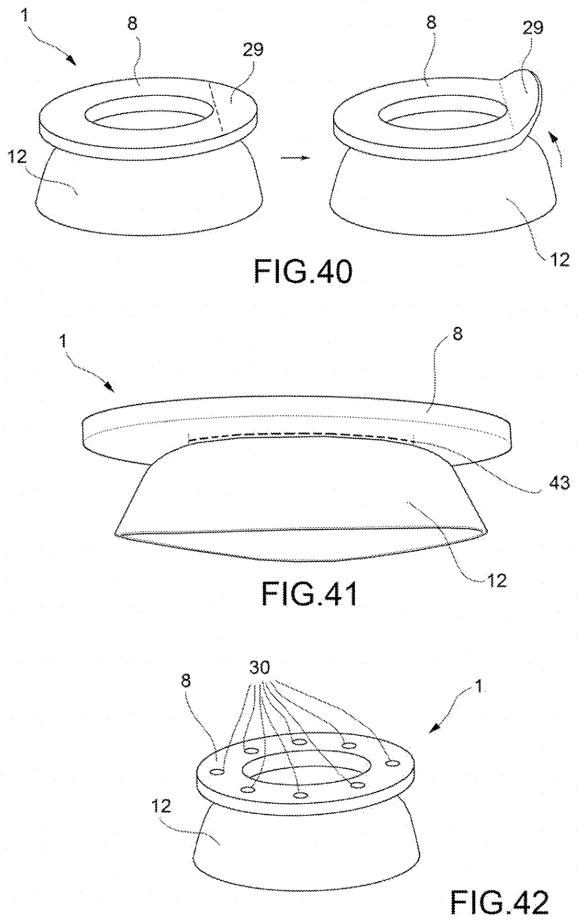

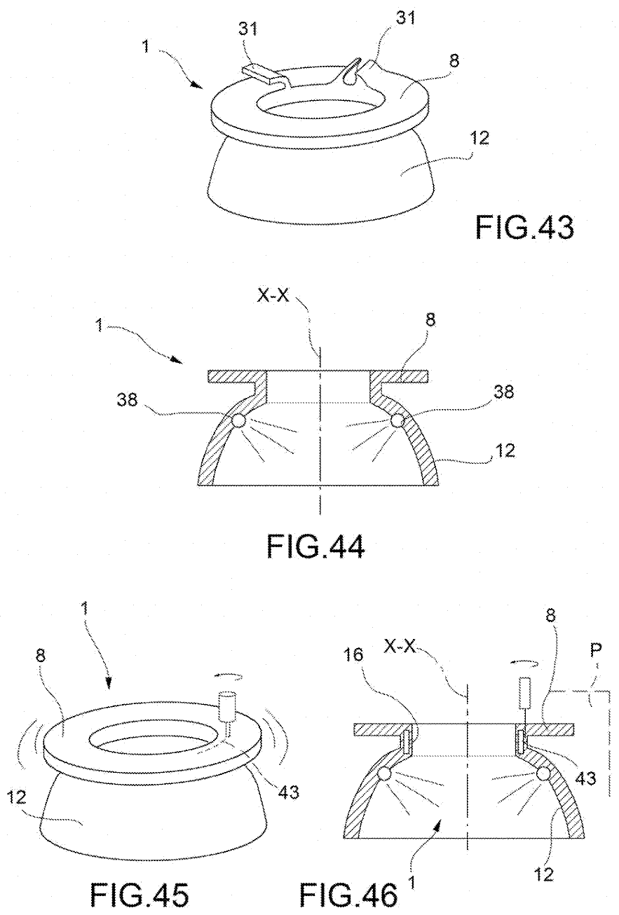

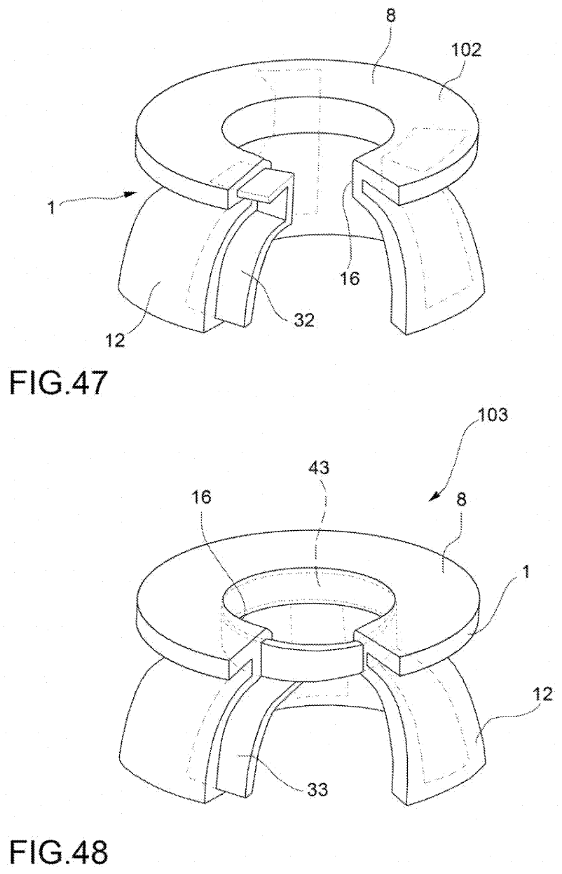

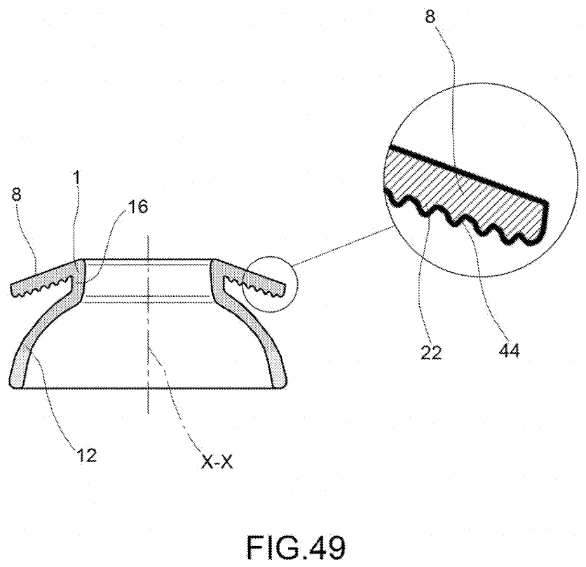

### DESCRIPTION OF SOME PREFERRED EMBODIMENTS

According to a general embodiment, a tissue retractor 1 for retracting incisions in cardiac tissues 100 is provided.

Said incisions 100 comprise an incision edge 101 of a tissue 104.

Said tissue retractor 1 comprises a retractor body 2.

Said retractor body 2 has a tubular shape which extends along a predetermined longitudinal direction X-X.

The term “tubular” means a body which surrounds a lumen, but not necessarily a continuous body which forms a closed wall.

The term “continuous tubular” means a body which surrounds a lumen and forms a continuous wall closed as a ring or annularly to support the entire perimeter of the incision edge.

Said retractor body 2 delimits a central device opening 3.

Said retractor body 2 is adapted to pass from a gathered or folded body position 4 to an extended or unfolded body position 5 in which it keeps said central device opening 3 pervious or open.

Said retractor body 2 in said extended or unfolded body position 5 extends so that, when said retractor body 2 is placed straddling said incision edge 101, it retracts said incision edge 101 by opening said tissue incision 100.

Said retractor body 2, with respect to said tissue incision 100, comprises a first proximal body end 6 and an opposite second distal body end 7.

Said first proximal body end 6 comprises a proximal flange

Said proximal flange 8 comprises an inner proximal flange edge 9 close to the longitudinal axis X-X and an outer proximal flange edge 10 opposite to the inner flange edge 9.

Said proximal flange 8 comprises a proximal flange body 11 which extends, with the device not delivered and thus not inserted into said tissue incision 100, in a cantilevered manner away from said central device opening 3.

Said second distal body end 7 comprises a distal flange

Said distal flange 12 comprises an inner distal flange edge 13 close to the longitudinal axis X-X and an outer distal flange edge 14 opposite to said inner distal flange edge 13.

Said distal flange 12 comprises a distal flange body 15 which extends, with the device not delivered, in a cantilevered manner away from said central device opening 3.

Said retractor body 2 further comprises a retractor neck

Said retractor neck 16 comprises a first proximal neck end 16 and an opposite second distal neck end 18.

Said first proximal neck end 16 is connected to said inner proximal flange edge 9.

Said second distal neck end 18 is connected to said inner distal flange edge 13.

Advantageously, said proximal flange 8 comprises at least one proximal flat or concave flange portion 19 having concavity facing the distal flange 12.

Said distal flange 12 comprises at least one distal concave flange portion 20 having concavity facing away from or towards the side opposite to said proximal flange 8, so that the concavity of the at least distal concave flange portion 20 and the concavity of the at least proximal flat or concave flange portion 19 are facing the same direction.

Said distal flange 12 is dome segment-shaped or spherical crown-shaped to support, once the retractor is delivered in the incision 101, the round shape of the bloodless atrial ceiling 40, which tends to collapse.

According to an embodiment, said at least one proximal flat or concave flange portion 19 extends to the entire said proximal flange 8.

According to an embodiment, said at least one proximal flat or concave flange portion 19 is a proximal flange 8 substantially entirely concave portion 19.

According to an embodiment, said at least one distal concave flange portion 20 is a distal flange 12 substantially entirely concave portion 20.

According to an embodiment, the proximal flange 8, said distal flange 12 and said retractor neck 16 are in one piece.

According to an embodiment, said retractor body 2 extends from a continuous tubular body.

According to an embodiment, said retractor body 2 extends to form a closed and continuous ring body to annularly support the edges or margins of the incision edges of the tissue incision.

According to an embodiment, said proximal flange 8 conforms over the tissue 104 when the device is delivered.

According to an embodiment, said proximal flat or concave flange portion 19 is adapted to anchor to the tissue margins 104 which delimit the incision edge 102.

According to an embodiment, said proximal flange 19 comprises at least the proximal flange surface 21 adapted to face said processed tissue 104.

According to an embodiment, said proximal flange 19 comprises at least the proximal flange surface 21 adapted to face said tissue 104 which comprises a proximal flange surface roughness facing said tissue 22, e.g. flange surface reliefs 44, such as grooves or knurls or indentations.

According to an embodiment, the distal concave flange portion 20 is a spherical cap or coining portion.

According to an embodiment, said distal concave flange portion 20 is adapted to open the surgical site.

According to an embodiment, said retractor neck 16 delimits said device central opening 3.

According to an embodiment, said retractor neck 16 has tubular neck walls.

According to an embodiment, said retractor neck 16 has a toroidal shape.

According to an embodiment, said retractor neck 16 has elasticity adapted to apply a retracting action adapted to contain the edges of the incision edge 101.

According to an embodiment, said retractor body 2 has an annular extension.

According to an embodiment, said proximal flange 8 has an annular disc-shaped extension.

According to an embodiment, said proximal flange 8 has a spherical-crown-shaped or spherical-cap-shaped extension with an opening inside it which defines said central device opening 3.

According to an embodiment, said distal flange 12 has a spherical crown or spherical cap extension with an opening inside it which defines said central device opening 3.

According to an embodiment, said retractor neck 16 has an annular extension.

According to an embodiment, said retractor neck 16 has a cylindrical extension.

According to an embodiment, said retractor body 2 comprises elastic material portions.

According to an embodiment, the retractor body 2 is made of elastic material, e.g. medical-grade silicone.

According to an embodiment, said proximal flange 8 is made of a first material different from the material of said distal flange 12.

According to an embodiment, said proximal flange 8 is made of a first material which is harder and/or less elastic than the material of said distal flange 12.

According to an embodiment, said proximal flange 8 is made of a first material different from the material of said retractor neck 16.

According to an embodiment, said proximal flange 8 is made of a first material different from the material of said distal flange 12 and of said material of said retractor neck 16.

According to an embodiment, said tissue retractor 1 is made by co-molding several of its portions 8, 12, 16 with materials of different elastic properties.

According to an embodiment, said proximal flange 8 has a proximal flange extension 23 considered on a radial-longitudinal plane P, which plane contains a radial direction R-R either orthogonal or substantially orthogonal to said longitudinal direction X-X and contains said longitudinal direction X-X; said proximal flange extension 23 extends from the inner proximal flange edge 9 and reaches the outer proximal flange edge 10.

According to an embodiment, said proximal flange extension is greater than the longitudinal neck extension 24 of the retractor neck 16, longitudinal neck extension 24 which goes from the proximal inner flange edge 9 to the distal inner flange edge 13.

According to an embodiment, said distal flange 12 has a distal flange extension 25 considered on a radial-longitudinal plane P, plane containing a radial direction R-R either orthogonal or substantially orthogonal to said longitudinal direction X-X and containing said longitudinal direction X-X; said distal flange extension 25 extends from the inner distal flange edge 13 and reaches the outer distal flange edge 14.

According to an embodiment, said distal flange extension 25 is greater than the proximal flange extension 23.

According to an embodiment, said proximal flange 8 has a non-uniform proximal flange extension 23 circumferentially turning about said longitudinal axis X-X, thus forming larger flange portions and smaller flange portions.

According to an embodiment, said distal flange 12 has a non-uniform distal flange extension 25 circumferentially turning about said longitudinal axis X-X, thus forming larger flange portions and smaller flange portions.

According to an embodiment, said distal flange 12 has a distal flange inflection portion 26 close to the inner distal flange edge 13 which forms an inflection with a first portion close to the inner distal flange edge 13 with concavity facing the proximal flange 8 and thus a second main portion further away from said inner distal flange edge 13 with opposite concavity, i.e. facing away from said proximal flange 8.

According to an embodiment, said distal flange 12 has an inflection portion 26 close to the inner distal flange edge 13 which forms a circumferential channel or inflection groove 27 between said distal flange 12 and said retractor neck 16.

According to an embodiment, said distal flange 12 comprises a flange body circumferentially extending in a continuous manner.

According to an embodiment, said distal flange 12 comprises a flange body having flange petal circumferentially extending in a continuous manner.

According to an embodiment, said central device opening 3 has a circular annular edge.

According to an embodiment, said central device opening 3 has an elliptical edge.

According to an embodiment, said central device opening 3 has an oval edge.

According to an embodiment, said central device opening 3 has a non-symmetrical edge.

According to an embodiment, said retractor body 2 comprises a body wall with a substantially constant thickness.

According to an embodiment, said retractor body 2 comprises a body wall with thinner portions 28.

According to an embodiment, said thinner portions 28 extend longitudinally and/or along radial planes P, so as to create predetermined folding lines of the retractor body 2. According to an embodiment, said thinner portions 28 are channels.

According to an embodiment, said proximal flange 8 comprises a thinner flange sector 29 adapted to be lifted by cardioplegia injection.

According to an embodiment, said retractor body 2 comprises flange windows 30 adapted to fix the flange 9 and/or 13 to the tissue or organ undergoing surgery or for surgery on a localized tissue prolapse or for the insertion of a surgical device.

According to an embodiment, the plurality of openings or flange windows 30 could be used, once the retractor is accommodated in the seat, to pass sutures then bringing them back to the exterior of the patient. By acting on these sutures, the surgeon can adjust the orientation of the heart with respect to the access point, like a kite guide, to optimize the exposure of the device and orientation.

According to an embodiment, the proximal flange 8 has at least one proximal flange ridge 31 on the surface thereof opposite to the tissue 104 which is adapted to attach or connect a device, such as a surgical device.

According to an embodiment, said proximal flange ridge 31 forms a connecting hook. According to an embodiment, said proximal flange ridge 31 forms a snap-on coupling seat.

According to an embodiment, said retractor body 3 comprises at least one portion thereof associated with a reinforcing element 32, e.g. adapted to locally increase the rigidity of the retractor to maintain said incision 100 retracted.

According to an embodiment, said reinforcing element 32 is made of metal material.

According to an embodiment, said reinforcing element 32 is made of shape-memory metal material.

According to an embodiment, said reinforcing element 32 is made of polymer material, e.g. reinforced polymer material.

According to an embodiment, said reinforcing element 32 is internally associated with said retractor body 3.

According to an embodiment, said reinforcing element 32 is embedded within said retractor body 3.

According to an embodiment, said reinforcing element 32 comprises a ring associated with said retractor neck 16.

According to an embodiment, said ring-shaped reinforcing element 32 is adjustable so as to adjust the size of the central device opening 4.

According to an embodiment, said ring-shaped reinforcing element 32 is adjustable as an elastic band with vertical adjustment.

According to an embodiment, said reinforcing element 32 comprise at least one reinforcing petal 33 which extends longitudinally.

According to an embodiment, said reinforcing petal 33 comprises a central reinforcing portion 34 adapted to associate with said retractor neck 16.

According to an embodiment, said central reinforcing part 34 comprises a petal seat 37 adapted to receive a reinforcing ring or reinforcing element 32.

According to an embodiment, said reinforcing petal 33 comprises a proximal reinforcing portion 35 adapted to associate with said proximal band 6. According to an embodiment.

According to an embodiment, said reinforcing petal 33 comprises a proximal reinforcing portion 35 adapted to associate with said proximal band 6.

According to an embodiment, said reinforcing petal 33 comprises a distal reinforcing portion 36 adapted to associate with said distal flange 12.

According to an embodiment, said at least one reinforcing petal 33 is a plurality of reinforcing petals 33.

According to an embodiment, said petal is adapted to position itself circumferentially with respect to said retractor body, said petal being placed in its preferred circumferential position either before creating the retractor body, in which case the reinforcing petal is embedded therein, or afterwards, in which case the reinforcing petal is placed inside the retractor body.

According to an embodiment, said plurality of reinforcing petals 33 are distributed circumferentially.

According to an embodiment, said plurality of reinforcing petals 33 are distributed equally distanced apart.

The present invention further relates to a tissue retractor 1 according to any one of the embodiments described above.

According to an embodiment, said tissue retractor 1 comprises a lighting device 38 adapted to illuminate the surgical site from the central device opening 4.

According to an embodiment, said lighting device 38 comprises a plurality of LEDs distributed circumferentially in said retractor body 3.

The present invention further relates to a surgical access device 102 comprising a tissue retractor 1 according to any one of the embodiments described above.

The present invention further relates to an atrial retractor 103 for cardiac surgery on mitral or tricuspid valve comprising a surgical access device 102 according to claim 14.

Those skilled in the art may make many changes and adaptations to the embodiments described above or may replace elements with others which are functionally equivalent in order to meet contingent needs without however departing from the scope of the appended claims.

According to an embodiment, the atrial retractor 103, comprises said tissue retractor 1, is generally annular (circular, elliptical) in shape, adapted to retract the margins of the incision 100 made in the atrial wall, or atrial ceiling 40, in order to expose the interior of atrium 41 in an optimal manner.

According to an embodiment, the tissue retractor 1 is made entirely of “rubbery” or elastic material, preferably of medical-grade silicone and with mechanical properties such as to give the required radial spreading force orthogonal to its longitudinal axis X-X to the retractor 1.

According to an embodiment, the tissue retractor 1 has a toroidal shape, with a “C”-shaped cross section but capable of following the convexity of the atrial ceiling 40, particularly adapted to contain the incision margins, which are embraced by the proximal flange 8 and the most proximal portion of the distal flange and contribute to supporting the retractor and keeping it in place.

According to an embodiment, the preferred general shape of flanges 8, 12 is that of a “dome segment” or “spherical crown”, to support the round shape of the atrial ceiling 40 once the retractor is delivered in the incision 100. More generally, the retractor 1 can be made with the branches of its “C”-shaped section either parallel or spread or converging or asymmetrical according to different angles, lengths and thicknesses, but always with the concavity of both flanges 8, 12 facing the same direction.

According to an embodiment, the flanges 8, 12 are annular and are created circumferentially by the branches of the “C”-shaped section. According to an embodiment, the flanges 8, 12 are continuous, or interrupted, or with variable thickness and profile. According to an embodiment, the flange inside the atrium or distal flange 12 can extend towards the atrium-ventricular septum to find support and stability.

According to an embodiment, the upper or proximal flanges 6 and the lower or distal flanges 12 structurally generate the main part of the radial force required to spread the incision 100, while the cylindrical connection portion or neck of retractor 16, mainly performs the function of containing the margins of the incision edge 101. According to an embodiment, the thickness and width of these body elements or portions 3 are the parameters which allow to determine the desired structural performance, together with the features of the material of the body 3.

According to an embodiment, the retractor 1 is reinforced with metal inserts or reinforcing elements 32, typically made of elastic or superelastic materials. According to an embodiment, such reinforcing elements 32 are inserted into the retractor neck 16, or on the proximal flange 6 and/or distal flange 12, or into one or more of said elements.

According to an embodiment, the retractor 1 may be associated with a retraction control system 43 if the surgeon deems it necessary to increase or decrease the exposure of the inner atrium.

According to an embodiment, this circumferential extension-adjustable reinforcing ring 43 is a metal ring or band, inserted into the retractor neck 16, which can be expanded circumferentially with respect to the basic configuration. Such an expansion can take place after positioning in situ, by acting directly with the fingers, or by adopting mechanisms inside the retractor, or by means of retractor accessories outside the retractor. The divisible metal element 43 can be made in a shape similar to common electrical ties or hose clamps, or in the shape of plastic deformable stents or the like.

According to an embodiment, the retractor 1 has a circular or elliptical or oval plan.

According to an embodiment, the proximal flange 8 has a circular or elliptical or drop-shaped plan.

According to an embodiment, the proximal flange 8 has either a constant width or a variable circumferential width.

According to an embodiment, the distal flange 12 is complete circumferentially or “scalloped” or perforated or with windows.

According to an embodiment, the retractor neck 16 has a reduced longitudinal extension equal to the thickness of the atrial wall and, according to an embodiment, forms a “pocket” throat with said distal flange to contain more atrial tissue.

According to an embodiment, the proximal flange 8 or distal flange 12 is non-homogeneous in order to respect the anatomical dimensions outside the atrium.

According to an embodiment, the distal flange 12 is scalloped to prevent occlusion of vessels which flow into the atrium.

According to an embodiment, the distal flange 12 is concave downwards (or away from the proximal flange) to minimize encumbrances around the working opening.

According to an embodiment, the retractor body 2 has a rubber spacer of its parts which is either constant or adapted locally to specific functional requirements, e.g.:

the proximal flange 8 is rather thick, to have sufficient force to create radial retraction of the incision margins;

the distal flange 12 has the thicker distal part, to have the force to retract the tissues surrounding the mitral valve.

According to an embodiment, there are thinner, therefore soft, areas in some points of the retractor 1 to promote the creation of predetermined and repetitive folds when contracting the retractor to load it, for example, on a delivery device or system.

According to an embodiment, the proximal flange 8 has a thinner, therefore less rigid, sector in contiguity with the aortic valve, so as not to alter the coaptation thereof and to maintain the possibility of administering the cardioplegia solution effectively without removing the retractor 1.

According to an embodiment, various types of reinforcing elements 32 (polymer, metal, spring, etc.) are inserted into the various parts of retractor 1 to increase its rigidity and/or retraction force:

rings or circumferential springs in the proximal flange 8, for greater expansion force;

and/or

reinforcing blades or petals 33 in the distal flange 12, for greater localized retraction force;

and/or

circumferential band in the neck of retractor 16 to maintain its circularity.

According to an embodiment, at least one body portion 2 has at least one of its surfaces with a smooth or rough or embossed or scored finish, either externally or internally, to meet specific functional needs, for example:

if the surface is in contact with the smooth inner tissues of the heart, for maximum atraumaticity;

if it is in contact with the wrinkly external heart surface to promote good friction on the tissues to be retracted.

According to an embodiment, the retractor 1 may be made using different materials to made specific parts of the product. Thereby, it is possible to optimize the type of material used according to the function of the part to be made. A typical example is the use of silicone rubbers of different hardness/elasticity for the proximal flange 8 and the distal flange.

According to an embodiment, the two flanges 8, 12 can be co-molded according to known techniques.

Another manner of simultaneous use of different materials may be to use reinforcing inserts inside the molded flange. An example may be that of a ring insert made of shape-memory material (plastic or metal) inside the retractor neck 16, to increase its radial force.

According to an embodiment, the reinforcing inserts can be co-molded according to known techniques.

According to an embodiment, the flange is pre-perforated to allow installation of service sutures.

According to an embodiment, the flange has seats for accessories (cannulas; illuminators; other) obtained in the flange itself.

According to an embodiment, LED illuminators are incorporated for better visualization of the surgical site in the retractor.

According to an embodiment, the retractor 1 is reinforced with a band ring 43 and a series of blades 33 mounted on the ring.

The band ring 43 is made of elastic materials (harmonic steel; technopolymers; composite materials) which convey the function of a spring capable of generating radial force thereto.

The blades 33, mounted on the ring 43 with mechanical solutions, fit between the margins of the atrial incision and achieve the retraction by virtue of the radial thrust produced by the band ring 43. They are made of metal or polymer materials.

The retractor 1 is mounted in the atrial incision by folding the ring 43 and the blades 33 therewith.

The blades 33 can be of various shapes.

The blades 33 can be mounted on ring 43 by the manufacturer, or can be chosen by the surgeon according to the number, shape, material and position considered most suited for optimal mitral exposure.

The blades 33 can slide to allow optimal placement on the field with respect to the patient's anatomy.

The ring 43 may have a fixed or adjustable diameter (e.g. pipe clamp type).

According to an embodiment, it is possible to mount other types of accessories on the ring, such as illuminators, supports for cannulas and vents, and the like.

According to an embodiment, the tissue retractor 1 is adapted to be delivered into a tissue incision 100 via a delivery system or retractor delivery system 105.

According to an embodiment, the tissue retractor 1 is adapted to be folded, e.g. by lifting or rotating the proximal flange 8 towards the longitudinal axis X-X, away from the distal flange 12 (see FIG. 7).

According to an embodiment, the tissue retractor 1, either unfolded or folded or folded on itself, can be loaded from the interior of the tubular body of the retractor delivery system 105, so as to insert the tubular body of the retractor delivery system 105 into the tissue incision 100, and then pushed out from the tubular body of the retractor delivery system 105 or released, e.g. by means of a pusher by retracting the tubular body of the retractor delivery system 105 as the tissue retractor 1 is made to exit by opening around the tissue incision 100, in particular by releasing or opening or extending its distal flange 12 below or within the incision 100, by placing the retractor neck 16 around the incision edge 101 and by opening or extending its proximal flange 8 above or outside the incision 100 (see FIGS. 13 to 18).

According to an embodiment, the tissue retractor 1 in its folded form is folded into a loop, then with a fold that takes a “U”-shape transversally to its longitudinal axis X-X (see FIG. 8).

According to an embodiment, the tissue retractor 1 in its folded form is folded into two loops, then with a fold that transversally to its longitudinal axis X-X takes a “S” shape (see FIG. 9).

According to an embodiment, the tissue retractor 1 in its folded form is folded into two opposite loops, thus with a fold that takes an “H” shape transversally to its longitudinal axis X-X (see FIG. 10).

According to an embodiment, one of the functions of tissue retractor 1 is to keep the edges of incision 101 of the atrial incision 100 made by the surgeon apart and open.

In particular, the margins of atrial tissue 104 immediately adjacent to the margins of the incision arrange themselves around the retractor neck 16. The longitudinal neck extension 24, formed between the proximal flange inner edge 9 and the distal flange inner edge 13, forms a “throat” which contains said atrial tissue margins, and these, to some extent, “pile up” in that space.

The wider the extension of the central device opening 3, e.g. the maximum extension or the diameter if it is circular, the more marked is the accumulation phenomenon of atrial tissues in the “throat”. It is therefore advantageous that, as the extension of the central device opening 3 increases, the longitudinal extension of neck 24 increases, to create a “throat” with more space to contain the tissues.

There is therefore a relationship of preferential proportionality between the longitudinal extension of neck 24 and the extension of the central device opening 3. In particular, the ratio of the longitudinal neck extension 24 to the extension of the central device opening 3 may typically vary in a range from ⅙ to ⅓.

According to an embodiment, a solution which can facilitate an easier housing of the margins of atrial tissue, with advantages in terms of atraumaticity, is to create a circumferential channel or inflection throat 27 close to the neck, e.g. as shown in FIG. 38.

By virtue of the solutions of tissue retractor 1, suggested in its various embodiments, it is possible to obtain one or more of the following advantages:

the increase of the longitudinal extension of neck 24 proportionally to the extension of the central device opening 3 promotes an always orderly arrangement of the separated tissues, thus reducing the stress on the margins of the incision 100;

the dome shape of the distal flange 12 optimizes the performance of the material, so that even a yielding and thin material such as silicone, which is thus atraumatic and foldable for insertion into a delivery system, has the structural properties required to ensure the support and effective retraction of the saggy atrial tissue of a bloodless heart subject to surgery;

the proximal flange 8, which is radially larger than the length of the longitudinal extension of the neck 24, provides the radial force required to keep the margins of the incision apart; if this function were entrusted to the radial thickness of the neck 16, the latter would have to be very thick, sacrificing in practice part of the free extension of the central device opening 3 to access the surgical site;

the distal flange 12, which is longer than the length of the neck 24, allows the creation of a wide dome which optimizes the size and shape obtained for the free surgical site.

According to a further embodiment, said proximal flange 8 has a proximal flange extension 23 (or path along the flange shorter than its inner edge and its outer edge) considered on a radial-longitudinal plane P, plane containing a radial direction R-R orthogonal or substantially orthogonal to said longitudinal direction X-X and containing said longitudinal direction X-X; said proximal flange extension 23 extends from the inner proximal flange edge 9 and reaches the outer proximal flange edge 10. Said proximal flange extension 23 is greater than the longitudinal (or axial) neck extension 24 of the retractor neck 16, longitudinal neck extension 24 which goes from the proximal inner flange edge 9 to the distal inner flange edge 13.

According to an embodiment, said distal flange 12 has a distal flange extension 25 (or path along the shorter flange from its inner edge and its outer edge) considered on a radial-longitudinal plane P, plane containing a radial direction R-R orthogonal, or substantially orthogonal to said longitudinal direction X-X, and containing said longitudinal direction X-X; said distal flange extension 25 extends from the inner distal flange edge 13 and reaches the outer distal flange edge 14. Said distal flange extension 25 is greater than the proximal flange extension 23.

By virtue of the suggested solutions, neither flange (distal nor proximal) is designed to provide sealing functions and/or an anti-ejection element of the device from the incision, because in the intended application of non-beating open-heart surgery these functions are unnecessary: the heart is still and empty, and the surrounding tissues are saggy.

In particular, the function of the two flanges (distal and proximal) is completely different from that of the flanges in WO 2011/017440 A2. In detail:

the proximal flange is designed to provide the radial force required to keep the margins of the incision made on the top of the atrium open; structurally, this result is obtained by working on its thickness and radial extension.

The distal flange or skirt or dome is instead designed to support the saggy tissues of the atrium (even in the gravitational sense: given the patient's typical position on the surgical bed, with an empty heart and an incised atrium, the saggy atrial tissues would tend to fall back into the atrium itself, effectively occupying the surgical site), and to keep them wide and out of the surgical site. The dome-shaped design of the distal flange is ideal for this purpose. It may be said that the dome works oppositely to the flange of WO 2011/017440 A2: the flange of WO 2011/017440 A2 must conform to the inner shapes of the heart; the distal flange of the invention should be as small as possible in shape: on the contrary, it is the saggy tissues of the atrium which, by resting on the distal flange, conform to it and are supported by it.

In the device which is the subject of the invention:

the proximal flange can be advantageously designed to be concave towards the distal flange for the sole purpose of minimizing the protrusion with respect to the atrial ceiling and the dimension towards the other elements of the surgical set-up (cannulas, optical fibers, various instruments);

the distal flange or skirt or dome has a concave shape facing the mitral valve for the reason inherent to its dome shape and its structural function of supporting the saggy tissues of the bloodless heart and moving away from the surgical site.

According to an embodiment, the device of the present invention differs from WO 2011/017440 A2 also by its size.

WO 2011/017440 A2 describes a valve access channel, i.e. an access port to the ventricle, in a range of diameters between 5 and 20 mm, while the solution suggested by the invention has a central device opening 3 with dimensions ranging between 30 and 50 mm.

In the embodiments in FIGS. 16 and 18 of WO 2011/017440 A2, flanges are shown having a maximum diameter which is approximately twice the longitudinal or axial length of the valve body. Considering a ventricular wall thickness of 10 mm, the flange diameters of WO 2011/017440 A2 would be 20-25 mm at most. According to an embodiment, the device 1 of the invention, the proximal flange has maximum radial diameters which can vary between 50 and 75 mm, as well as the distal flange.

In the embodiments in FIGS. 16 and 18 of WO 2011/017440 A2, the distal flange is always shown flat, i.e. with an axial height of 0. According to an embodiment, the device 1 of the invention, the non-deformed distal flange has an axial height ranging between 15 and 45 mm.

In embodiments in FIGS. 16 and 18 of WO 2011/017440 A2, the dimensional ratio between the outer diameters of the proximal and distal flanges and the length of the central body or port appears not to exceed 2.5 times. According to an embodiment, the device 1 of the invention, considering a maximum length of 8-10 mm for the neck, said ratio can reach 7-8 times.

According to an embodiment, said distal flange 12 has a distal flange geometry and thickness which constitute a supporting structure and such as to retract and support the cardiac tissues 104 thus creating a free surgical site.

According to an embodiment, the ratio of the longitudinal neck extension 24 to the central device opening extension 3 is in the range from ⅙ to ⅓.

According to an embodiment, said proximal flange 8 has an extension, meaning the distance or path between the inner proximal flange edge 9 thereof and its outer proximal flange edge 10, which is greater than the longitudinal neck extension 24.

According to an embodiment, said distal flange 12 has an extension, meaning the distance or path between the inner distal flange edge 13 thereof and its outer distal flange edge 14, which is greater than the longitudinal neck extension 24.

According to an embodiment, said distal flange 12 has an extension, meaning the distance or path, between the inner distal flange edge 13 thereof and its outer distal flange edge 14, which is greater than the proximal flange extension of said proximal flange 8, meaning the distance or path, between its inner proximal flange edge 9 and its outer proximal flange edge 10.

According to an embodiment, said distal flange 12, with the device not delivered, protrudes away from said retractor neck 16, thus increasing its radial extension in a less than linear manner to form a dome.

According to an embodiment, said distal flange 12, with the device not delivered, protrudes away from said retractor neck 16, thus forming an angle with said retractor neck 16 between 90 DEG and 75 DEG.

According to an embodiment, said proximal flange 8 has a distal flange inflection portion close to the inner proximal flange edge 9 which forms a inflection with a first portion proximal to the inner proximal flange edge 9 with concavity facing the distal flange and thus a second main portion further away from said inner proximal flange edge 9 with opposite concavity, i.e. facing away from said distal flange 12.

According to an embodiment, said proximal flange 8 has an inflection portion 26 close to the inner proximal flange edge 9 which forms a circumferential channel or inflection groove between said proximal flange 8 and said retractor neck 16.

By virtue of the suggested solutions, it is possible to optimize the shape and size of the surgical site, and to do so in a completely atraumatic manner. In order to achieve this, soft materials, such as silicone or polyurethane are used, but these would not ensure the force required to retract if they were applied to known architectures. The innovative design of the device 1 of the invention solves the problem by implementing a double retraction action:

the proximal flange, which is almost flat and relatively thick, provides the radial force required to circularly spread the margins of the incision;

the distal flange or skirt or dome, thin to minimize the overall dimensions, broadly supports the soft tissues of the atrium from the interior, which tissues lean on it at 360° and take the shape thereof. And it is precisely the dome shape which geometrically allows to obtain a wide surgical site free from encumbrances and structurally allows a thin layer of silicone to have the strength required to support the atrial tissues.

Furthermore, the two flanges are connected by a neck, also made of silicone, axially only as long as the walls of the atrial ceiling are thick, around which the margins of the incision are located and rest in a completely atraumatic manner.

### LIST OF REFERENCES

1 tissue retractor
2 retractor body
3 central device opening
4 gathered or folded body position
5 extended or unfolded body position
6 first proximal body end
7 second distal body end
8 proximal flange
9 inner proximal flange edge
10 outer proximal flange edge
11 proximal flange body
12 distal flange
13 inner distal flange edge
14 outer distal flange edge
15 distal flange body
16 retractor neck
17 first proximal neck end
18 second distal neck end
19 flat or concave proximal flange portion
20 distal concave flange portion
21 proximal flange surface
22 proximal flange surface roughness facing said tissue
23 proximal flange extension
24 longitudinal neck extension
25 distal flange extension
26 distal flange inflection portion
27 circumferential channel or inflection throat
28 thinner portions or longitudinal channels
29 flange sector
30 flange windows
31 proximal flange ridge
32 reinforcing element
33 reinforcing petal
34 central reinforcing portion
35 proximal reinforcing portion
36 distal reinforcing portion
37 petal seat
38 lighting device
39 heart
40 atrial ceiling
41 atrium
42 ventricle
43 reinforcing ring being adjustable in circumferential extension
100 cardiac tissue incisions
101 incision edge
102 surgical access device
103 atrial retractor
104 heart tissue
105 delivery system retractor
X-X longitudinal extension axis of the device
R-R radial direction orthogonal to said longitudinal direction
P radial-longitudinal plane or radial plane

What is claimed is:

1-62. (canceled)

63. A tissue retractor for retracting a tissue incision and supporting cardiac tissue of an atrial ceiling, wherein said tissue incision comprises an incision edge, and wherein
said tissue retractor comprises a retractor body, wherein:
said retractor body has a tubular shape extending along a predetermined longitudinal axis direction;
said retractor body delimits a central device opening;
said retractor body is adapted to pass from a gathered or folded body position to an extended or unfolded body position in which it keeps said central device opening pervious;
said retractor body in said extended or unfolded body position extends so that, when said retractor body is placed straddling said incision edge, it retracts said incision edge by opening said tissue incision;
said retractor body with respect to said tissue incision comprises a first proximal body end and an opposite second distal body end;
said first proximal body end comprises a proximal flange;
said proximal flange comprises an inner proximal flange edge close to the longitudinal axis and an outer proximal flange edge opposite to the inner proximal flange edge;
said proximal flange comprises a proximal flange body which extends, with the tissue retractor not delivered, in a cantilevered manner away from said central device opening;
said second distal body end comprises a distal flange;
said distal flange comprises an inner distal flange edge proximal to the longitudinal axis and an outer distal flange edge opposite to said inner distal flange edge;
said distal flange comprises a distal flange body which extends, with the tissue retractor not delivered, in a cantilevered manner away from said central device opening;
said retractor body further comprises a retractor neck;
said retractor neck comprises a first proximal neck end and an opposite second distal neck end;
said first proximal neck end is connected to said inner proximal flange edge;
said second distal neck end is connected to said inner distal flange edge;
said proximal flange comprises at least one proximal flat or concave flange portion having concavity facing said distal flange;
said distal flange comprises at least one distal concave flange portion having concavity either facing away from or towards a side opposite to said proximal flange, so that the concavity of the at least one distal concave flange portion and the concavity of the at least one proximal flat or concave flange portion are facing a same direction; and wherein
the distal flange is dome segment-shaped or spherical crown-shaped to support, once the tissue retractor has been delivered in the tissue incision, a round shape of the bloodless atrial ceiling, which tends to collapse.

64. The tissue retractor of claim 63, wherein said distal flange has a distal flange geometry and thickness which constitute a supporting structure to retract and support the cardiac tissue, thus creating a free surgical site.

65. The tissue retractor of claim 63, wherein said distal flange, with the tissue retractor not delivered, protrudes away from said retractor neck, thus increasing its radial extension in a less than linear manner to form a dome.

66. The tissue retractor of claim 63, wherein the proximal flange, the distal flange and the retractor neck are in one piece.

67. The tissue retractor of claim 63, wherein said retractor body extends to form a closed and continuous ring body to annularly support edges or margins of the incision edge.

68. The tissue retractor of claim 63, wherein, when the tissue retractor is delivered, the proximal flange conforms over the cardiac tissue.

69. The tissue retractor of claim 63, wherein said at least one distal concave flange portion is adapted to open the surgical site.

70. The tissue retractor of claim 63, wherein said retractor neck has elasticity adapted to apply a retracting action to contain the edges of the incision edge.

71. The tissue retractor of claim 63, wherein said retractor body is made of elastic material, comprising medical-grade silicone.

72. The tissue retractor of claim 63, wherein said proximal flange is made of a material different from the material of said distal flange.

73. The tissue retractor of claim 63, wherein said tissue retractor is made by co-molding several of different portions thereof with materials of different elastic properties.

74. The tissue retractor of claim 63, wherein said distal flange has an inflection portion close to the inner distal flange edge which forms a circumferential channel or inflection throat between said distal flange and said retractor neck.

75. The tissue retractor of claim 63, wherein said proximal flange has an inflection portion close to the inner proximal flange edge which forms a circumferential channel or inflection throat between said proximal flange and said retractor neck.

76. The tissue retractor of claim 63, wherein said retractor body comprises a body wall with thinner portions, wherein said thinner portions extend longitudinally and/or along radial planes to create predetermined folding lines of the retractor body, and/or wherein said thinner portions are channels.

77. The tissue retractor of claim 63, wherein said retractor body comprises at least one portion associated with a reinforcing element adapted to locally increase rigidity of the retractor body to maintain said incision retracted, wherein
said reinforcing element is made of metal material, and/or wherein
said reinforcing element is made of shape-memory metal material, and/or wherein
said reinforcing element is made of reinforced polymer material.

78. The tissue retractor of claim 77, wherein
said reinforcing element is internally associated with said retractor body; and/or wherein
said reinforcing element is embedded within said retractor body; and/or wherein
said reinforcing element comprises a ring associated with said retractor neck, wherein
said ring-shaped reinforcing element is adjustable to adjust a size of the central device opening, and/or wherein
said ring-shaped reinforcing element is adjustable as an elastic band with vertical adjustment; and/or wherein
said reinforcing element comprises at least one reinforcing petal extending longitudinally, wherein
said at least one reinforcing petal comprises a central reinforcing portion adapted to associate with said retractor neck, wherein said central reinforcing portion comprises a petal seat adapted to receive a reinforcing ring or reinforcing element; and/or wherein
said at least one reinforcing petal comprises a proximal reinforcing portion adapted to associate with said first proximal body end; and/or wherein
said at least one reinforcing petal comprises a distal reinforcing portion adapted to associate with said distal flange; and/or wherein
said at least one reinforcing petal is a plurality of reinforcing petals, wherein said plurality of reinforcing petals are distributed circumferentially, and/or wherein said plurality of reinforcing petals are distributed equally distanced apart.

79. A surgical access device comprising at least one tissue retractor for retracting a tissue incision and supporting cardiac tissue of an atrial ceiling, wherein said tissue incision comprises an incision edge, and wherein
said tissue retractor comprises a retractor body, wherein:
said retractor body has a tubular shape extending along a predetermined longitudinal axis direction;
said retractor body delimits a central device opening;
said retractor body is adapted to pass from a gathered or folded body position to an extended or unfolded body position in which it keeps said central device opening pervious;
said retractor body in said extended or unfolded body position extends so that, when said retractor body is placed straddling said incision edge, it retracts said incision edge by opening said tissue incision;
said retractor body with respect to said tissue incision comprises a first proximal body end and an opposite second distal body end;
said first proximal body end comprises a proximal flange;
said proximal flange comprises an inner proximal flange edge close to the longitudinal axis and an outer proximal flange edge opposite to the inner proximal flange edge;
said proximal flange comprises a proximal flange body which extends, with the tissue retractor not delivered, in a cantilevered manner away from said central device opening;
said second distal body end comprises a distal flange;
said distal flange comprises an inner distal flange edge proximal to the longitudinal axis and an outer distal flange edge opposite to said inner distal flange edge;
said distal flange comprises a distal flange body which extends, with the tissue retractor not delivered, in a cantilevered manner away from said central device opening;
said retractor body further comprises a retractor neck;
said retractor neck comprises a first proximal neck end and an opposite second distal neck end;
said first proximal neck end is connected to said inner proximal flange edge;
said second distal neck end is connected to said inner distal flange edge;
said proximal flange comprises at least one proximal flat or concave flange portion having concavity facing said distal flange;
said distal flange comprises at least one distal concave flange portion having concavity either facing away from or towards a side opposite to said proximal flange, so that the concavity of the at least one distal concave flange portion and the concavity of the at least one proximal flat or concave flange portion are facing a same direction; and wherein
the distal flange is dome segment-shaped or spherical crown-shaped to support, once the tissue retractor has been delivered in the tissue incision, a round shape of the bloodless atrial ceiling, which tends to collapse.

80. An atrial retractor for cardiac surgery on mitral or tricuspid valve comprising the surgical access device of claim 79.

81. An assembly comprising
a tissue retractor for retracting a tissue incision and supporting cardiac tissue of an atrial ceiling, wherein said tissue incision comprises an incision edge, and wherein
said tissue retractor comprises a retractor body, wherein:
said retractor body has a tubular shape extending along a predetermined longitudinal axis direction;
said retractor body delimits a central device opening;
said retractor body is adapted to pass from a gathered or folded body position to an extended or unfolded body position in which it keeps said central device opening pervious;
said retractor body in said extended or unfolded body position extends so that, when said retractor body is placed straddling said incision edge, it retracts said incision edge by opening said tissue incision;
said retractor body with respect to said tissue incision comprises a first proximal body end and an opposite second distal body end;
said first proximal body end comprises a proximal flange;
said proximal flange comprises an inner proximal flange edge close to the longitudinal axis and an outer proximal flange edge opposite to the inner proximal flange edge;
said proximal flange comprises a proximal flange body which extends, with the tissue retractor not delivered, in a cantilevered manner away from said central device opening;
said second distal body end comprises a distal flange;
said distal flange comprises an inner distal flange edge proximal to the longitudinal axis and an outer distal flange edge opposite to said inner distal flange edge;
said distal flange comprises a distal flange body which extends, with the tissue retractor not delivered, in a cantilevered manner away from said central device opening;
said retractor body further comprises a retractor neck;
said retractor neck comprises a first proximal neck end and an opposite second distal neck end;
said first proximal neck end is connected to said inner proximal flange edge;
said second distal neck end is connected to said inner distal flange edge;
said proximal flange comprises at least one proximal flat or concave flange portion having concavity facing said distal flange;
said distal flange comprises at least one distal concave flange portion having concavity either facing away from or towards a side opposite to said proximal flange, so that the concavity of the at least one distal concave flange portion and the concavity of the at least one proximal flat or concave flange portion are facing a same direction; and wherein
the distal flange is dome segment-shaped or spherical crown-shaped to support, once the tissue retractor has been delivered in the tissue incision, a round shape of the bloodless atrial ceiling, which tends to collapse, and
a delivery system, wherein said delivery system comprises a tubular body into which said tissue retractor is inserted, said tubular body being adapted to be inserted into the tissue incision to release or lay said tissue retractor across the incision edge of said tissue incision.

82. The assembly of claim 81, wherein said tissue retractor is accommodated folded in said delivery system, thus forming a shape with “H”-shaped folded loops in a section thereof transversal to its longitudinal extension.
